(19) United States
(12) Patent Application Publication
(10) Pub. No.: US 2019/0374410 A1
(43) Pub. Date: Dec. 12, 2019

(54) DIAPER WITH URINARY TRACT NOTIFICATION STRIP

(71) Applicant: Adil Arabbo, Troy, MI (US)

(72) Inventor: Adil Arabbo, Troy, MI (US)

(21) Appl. No.: 16/434,721

(22) Filed: Jun. 7, 2019

Related U.S. Application Data
(60) Provisional application No. 62/683,390, filed on Jun. 11, 2018.

(51) Int. Cl. A61F 13/84; A61F 13/49; A61B 5/20; A61B 5/00; A61B 5/1477; A61B 5/145

(52) U.S. Cl. CPC A61F 13/84; A61F 13/49; A61B 5/20; A61F 2013/422; A61B 5/1477; A61B 5/14507; A61B 5/6808

(57) ABSTRACT

A diaper incorporating at least one urine reactant for indicating the existence of a positive condition not limited to diagnosing of a urinary tract infection. The urine reactant further comprising a plurality of chemical agents or indicators incorporated into a test strip. Additional features can include the test strip being integrated into either an interior or exterior of a body of the diaper. In this fashion, and upon a wearer urinating onto the test strip and upon the agents/indicators each in response providing a positive indication, an output is provided which alerts the patient or caregiver as to the possibility of a urinary tract infection.

### CROSS REFERENCE TO RELATED APPLICATIONS

The present application claims the priority of U.S. Ser. No. 62/683,390 filed Jun. 11, 2018.

### FIELD OF THE INVENTION

The present invention discloses a diaper, typically for use by disabled adults or the elderly. More particularly, the present invention teaches a diaper incorporating a urine strip (internal or front located) which includes a plurality of chemical agents or indicators, these incorporated into a test strip. Upon the patient urinating onto the test strip, and upon the agents/indicators each providing a positive, an output is provided which alerts the patient or caregiver as to the possibility of a urinary tract infection and which would result in either treatment if the patient is symptomatic or further medical work up if necessary.

### BACKGROUND OF THE INVENTION

Relevant medical statistics suggest that urinary tract (UTI) infections account for approximately thirty-six percent of infections recorded in hospitalized patients. Early detection and treatment of an underlying UTI is further known to result in decreased hospital admissions with associated reduction in Medicare costs by billions of dollars annually. Given further that most patients that are dependent on the use of adult briefs are required to be changed 3-4 times per day, this frequency will provide for early detection and treatment of UTI's.

Examples of methods, devices and compositions for diagnosing, predicting and monitoring a urinary tract infection can alternatively include detecting of HD5 in the urine o a subject as set forth in Haim US 2014/0322722 and US 2016/0069905. Related WO 2016/161413 also teaches UTI detection via determining the level of human α-defensin 5 (HD5) and/or human neutrophil peptides (HNP) 1-3 in the urine sample and comparing it to a corresponding control value; and diagnosing the subject as having a urinary tract infection if the level of HD5 and/or HNP1-3 is greater than the control value. Kits for diagnosing a urinary tract infection in a subject using antibodies specific for HD5 and HNP1-3 are also described.

### SUMMARY OF THE PRESENT INVENTION

The present invention discloses a diaper incorporating at least one urine reactant for indicating the existence of a positive condition not limited to diagnosing of a urinary tract infection. The urine reactant further comprising a plurality of chemical agents or indicators incorporated into a test strip.

Additional features can include the test strip being integrated into either an interior or exterior of a body of the diaper. In this fashion, and upon a wearer urinating onto the test strip and upon the agents/indicators each in response providing a positive indication, an output is provided which alerts the patient or caregiver as to the possibility of a urinary tract infection.

The output may further include a colorant not limited to red which is released by instruction of the test strip in order to notify the wearer or care giver. Additional features include the test strip defining a first layer and a second strip defining a second layer sandwiching opposite sides of the diaper body which further integrates a detection material constructed to react to a chemical conduit established between the layers.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is an environmental illustration of the diaper according to the present invention with a forward positioned and internally embedded urine strip;

FIG. 2 is a similar illustration of a diaper according to a further variant in which the urine strip is located on an external forward facing location of the diaper;

FIGS. 3 and 4 illustrate respective side views of FIGS. 1-2;

FIG. 5 is a cutaway view taken along line 5-5 of FIG. 1 and depicting a direction of urine coating the internally positioned strip;

FIG. 6 is a corresponding cutaway view taken along line 6-6 of FIG. 2 and depicting a direction of urine coating the externally positioned strip;

FIG. 7 is a succeeding view to FIG. 6 and illustrating detachability of the externally supported urine test strip;

FIG. 8 is a flow chart providing an example of a plurality of activating agents or indicators, such including any of Nitrites, Leukocytes, Bacteria, pH Level Indicators, Red Blood Cell indicators, which are located within an embedded test strip and which, upon contacted by the urine, are each tested and, if most or all test positive, provide a positive indicator output (such without limitation including a red colorization identifier) in order to visually confirm the existence of a bladder infection;

FIGS. 9A-9B provide an example of a further variant of diaper, as viewed in cutaway along line 9-9 of FIG. 1, and combining both an inner test strip layer and an outer visual indicating layer separated by a detection materials integrated into the diaper sandwiched between the inner and outer layers; and

FIG. 10 is a flow chart similar to that depicted in FIG. 8 and providing further indication of interposed detection materials reacting to a chemical conduit established between the inner and outer layers, such providing (upon positive triggering of each chemical identifier or agent) a visual color reading on the outer layer that represents communication of the urine agent and that urine results are detected, this further including visual confirmation of existence of the conditions for a bladder infection.

### DETAILED DESCRIPTION OF THE PREFERRED EMBODIMENTS

With reference to FIGS. 1-10, the present invention discloses a diaper, typically for use by disabled adults or the elderly. More particularly, the present invention teaches a diaper incorporating a urine strip (internal or front located) which includes a plurality of chemical agents or indicators, these incorporated into a test strip. Upon the patient urinating onto the test strip, and upon the agents/indicators each providing a positive, an output is provided which alerts the patient or caregiver as to the possibility of a urinary tract infection and which would result in either treatment if the patient is symptomatic or further medical work up if necessary.

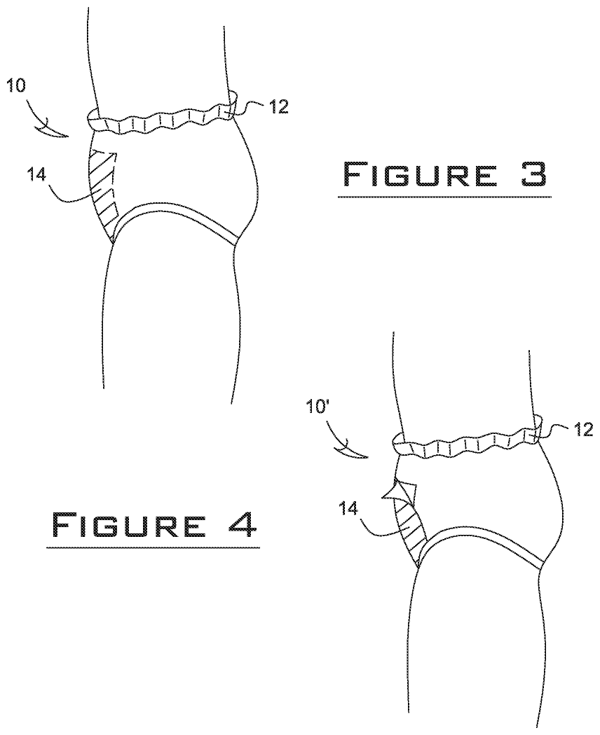

Referring initially to FIG. 1, an environmental illustration of the diaper according to the present invention is depicted at 10 according to any suitable construction, such including a conforming body of such as a cotton or other suitable, including synthetic, fluid absorbent material and having a waistband 12 encircling an upper edge. As also shown, in the side view of FIG. 3, a forward positioned and internally embedded urine strip 14 is provided internally of the diaper in proximity to a path of urine associated with a patient/wearer of the strip (male or female) and such as being further shown in an intermediate extending fashion between a front and back of the diaper at a location corresponding to a urinary orifice of the patient.

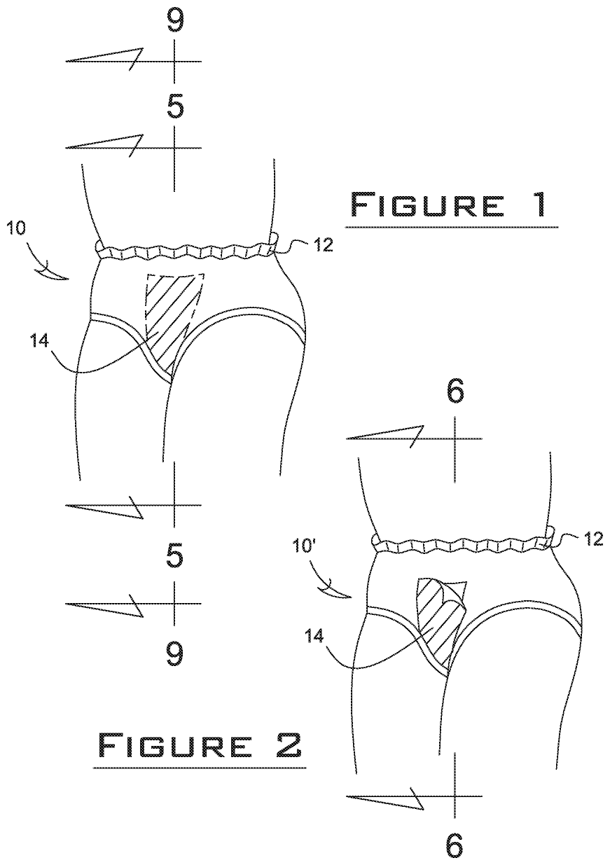

FIG. 2 is a similar illustration of a diaper according to a further variant 10′ in which the urine strip 14 is instead located on an external forward facing location of the diaper. As also shown in the side view of FIG. 4, the strip 14 can be positioned such that it is still in fluidic communication with the interior of the diaper (such including the provision of pathways integrated into the diaper material for communicating an adequate amount of fluid to the test strip).

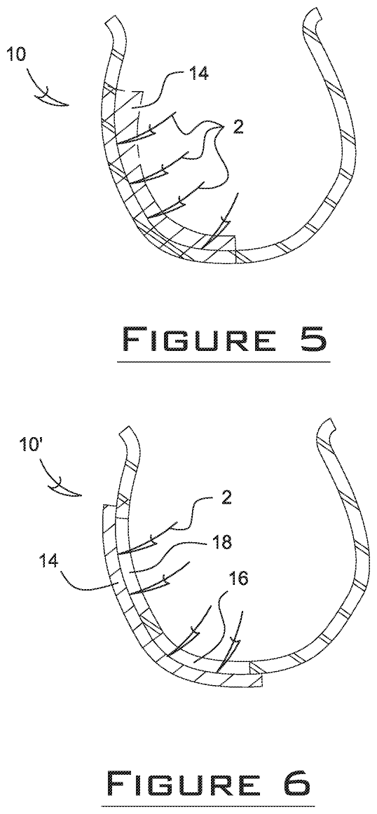

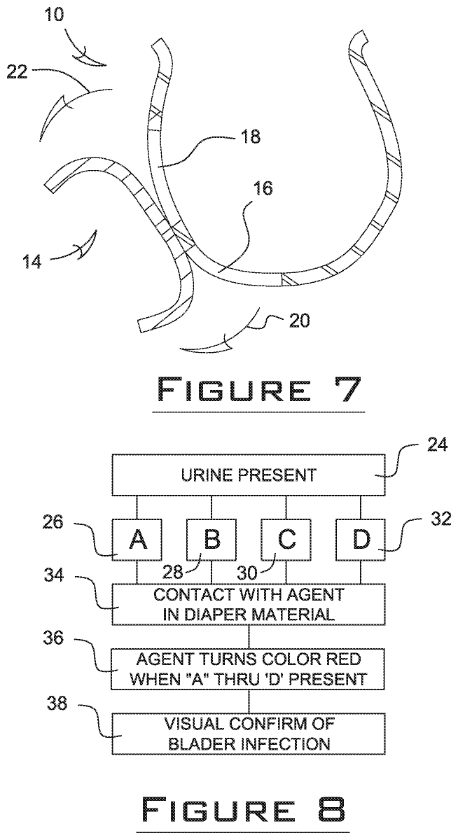

FIG. 5 is a cutaway view taken along line 5-5 of FIG. 1 and depicting a direction of urine 2 coating the internally positioned strip 14. FIG. 6 is a corresponding cutaway view taken along line 6-6 of FIG. 2 and depicting a direction of urine, again at 2, coating the externally positioned strip 14 and such further utilizing the existence of fluid communicating pathways (see at 16 and 18 which can also represent reduced thickness or varying material locations of the diaper which ensure travel of adequate urine to the exteriorly located test strip). FIG. 7 is a succeeding view to FIG. 6 and illustrating detachability of the externally supported urine test strip 14, such peeling away or otherwise attaching such as with the use of hook and loop (also Velcro) portions (not shown) and as referenced by directional arrows 20 and 22.

FIG. 8 is a flow chart of the operating protocol for providing an output indicative of a UTI condition. A representation of urine being present (at 24) is communicated to a plurality of individual activating agents or indicators, these represented at 26, 28, 30 and 32 and including, without limitation, any of Nitrites, Leukocytes, Bacteria, pH Level Indicators, Red Blood Cell indicators or the like.

As understood, the agents/indicators 26-32 are embedded or otherwise integrated into the test strip and, upon being contacted by the urine (step 34), are each tested by reacting with the urine. In the event that most or (typically) all of the indicators test positive (at step 36), a positive indicator output is provided, such without limitation including a visual identifier at 38 of a red or other colorization notifying the patient or caregiver of the indication of a UTI.

Although not shown in this variant, the present invention contemplates the incorporation of a separate fluidic sleeve or vial containing a volume of the red (colorant) liquid, such integrated in upon the diaper in proximity to the test strip. The separate fluidic sleeve can include a cap or other closure which is triggered by an output of the test strip. In one non-limiting instance, this can include the release of an agent for dissolving a closure of the die colorant and which, upon being activated, quickly colors the area of the diaper in a manner to notify the existence of conditions indicative of a UTI infection. Other envisioned variants can include the use of a die colorant powder incorporated into the diaper and which, upon the test strip again providing a collective positive output, introducing a further reactant into proximity with the powder (such including any of a separate fluid or an agent which can mix with the urine present in the diaper) and in order to provide an effective positive result indication.

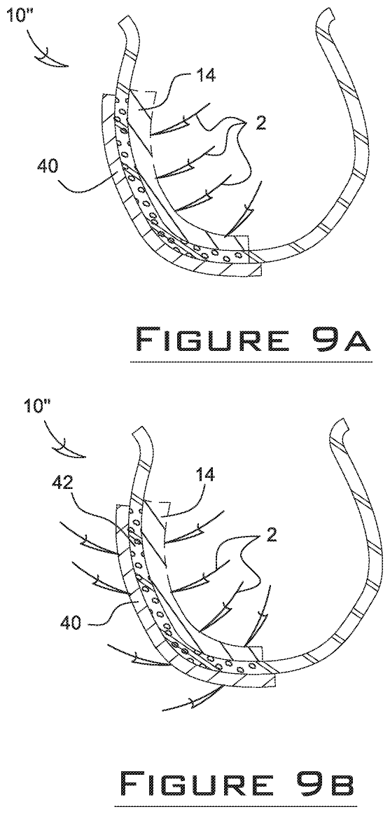

FIGS. 9A-9B provide an example of a further variant of diaper, depicted at 10″, and which combines both an inner test strip layer 14 and an outer visual indicating layer (see further at 40), these separated by a detection material (at 42) integrated into the diaper and sandwiched between the inner 14 and outer 40 layers.

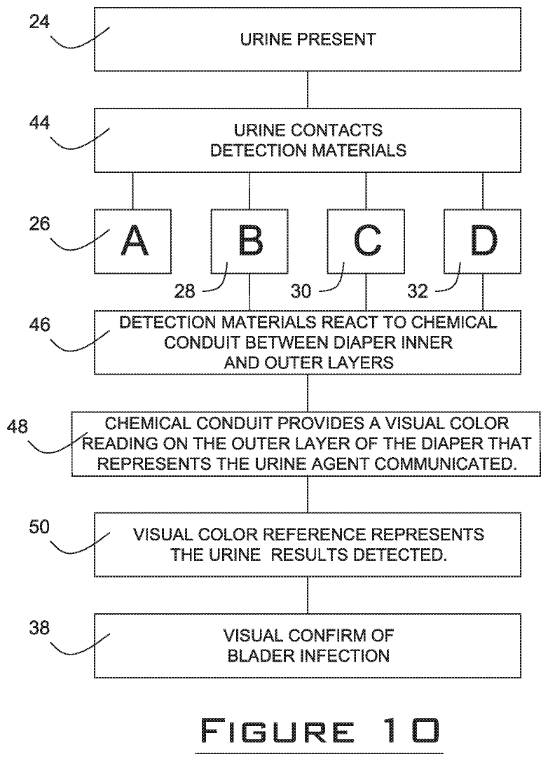

FIG. 10 is a flow chart similar to that depicted in FIG. 8, with identical steps being identically numbered at 24, 26-32 and 38, and providing further indication of interposed detection materials reacting to a chemical conduit (following contact of the urine at step 44 with the chemical identifiers 26-32 and reaction of the detection materials at 46). The detection materials (see also at 42 in FIGS. 9A-9B) are again established between the inner 14 and outer 40 layers and, upon chemical conduit providing a visual color reading on the outer layer 40 (at step 48), representing to the viewer that the urine agent was communicated.

Upon providing (upon positive triggering of each chemical identifier or agent) a visual color reading on the outer layer (step 50), a representation of the communication of the urine agent and that urine results are detected is provided, this further including visual confirmation of existence of the conditions for a bladder infection (step 38).

Other considerations include false positive test results occurring due to urine not being collected as a clean catch or in a sufficiently sterile fashion. That said, the urine strip serves as a reminder to the patient and care giver to monitor the strip and, upon receiving a visual positive indication, to provide follow up treatment or medical work up.

Having described my invention, other and additional preferred embodiments will become apparent to those skilled in the art to which it pertains, and without deviating from the scope of the appended claims. The detailed description and drawings are further understood to be supportive of the disclosure, the scope of which being defined by the claims. While some of the best modes and other embodiments for carrying out the claimed teachings have been described in detail, various alternative designs and embodiments exist for practicing the disclosure defined in the appended claims.

This can include the urine activating agents being integrated into other than a test strep and such as directly into the diaper or other otherwise. The detection protocols in the invention can further be revised to potentially diagnose other conditions not limited to UTI's, such including potentially other urinary type conditions and/or conditions associated with the wearers feces.

Other and additional envisioned embodiments can further include remote sensors which can be incorporated into the patient worn test strip and which, upon the designated reactants having been triggered, causing a separate colorant indicator (this can further include without limitation such as a colored die contained within any of a conduit, pouch, or frangible reservoir) to be released to thereby indicate a positive reading. Without limitation, the remote sensors can include any type of miniaturized communication device such as including a wireless (Bluetooth, Near Field Communication, WiFi, etc.) enabled transmitter which is incorporated into the sensor or other location of the diaper. In this manner, and upon the test strip located sensor determining a positive condition, the transmitter issues an output signal to any remote process enabled device (such as to a care provider tablet or smartphone having a mobile application or other program interfacing with the transmitter) to notify of the patient condition.

I claim:

1. An infection diagnosing diaper adapted to being worn by a patient, comprising:
a urine reactant incorporated into a test strip which is integrated in the diaper for indicating the existence of a positive condition not limited to diagnosing of a urinary tract infection; and
the urine reactant including at least one chemical agent or indicator and which, upon coming into contact with urine issued by the patient and triggering the positive condition, alerting a care provider of the condition.

2. The invention as described in claim 1, further comprising said test strip being integrated into either an interior or exterior of a body of the diaper.

3. The invention of claim 1 said test strip being positioned in extending fashion along a midsection of the diaper.

4. The invention as described in claim 1, said output further including a colorant which is released by instruction of the test strip in order to notify the wearer or care giver.

5. The invention as described in claim 1, further comprising the test strip defining a first layer and a second strip defining a second layer sandwiching opposites sides of the diaper body which further integrates a detection material constructed to react to a chemical conduit established between the layers.

6. An infection diagnosing diaper adapted to being worn by a patient, comprising:
a urine reactant incorporated into a test strip which is integrated in the diaper in extending fashion along a midsection of the diaper for indicating the existence of a positive condition not limited to diagnosing of a urinary tract infection; and
the urine reactant including at least one chemical agent or indicator and which, upon coming into contact with urine issued by the patient and triggering the positive condition, alerting a care provider of the condition.

7. The invention as described in claim 6, further comprising said test strip being integrated into either an interior or exterior of a body of the diaper.

8. The invention as described in claim 6, said output further including a colorant which is released by instruction of the test strip in order to notify the wearer or care giver.

9. The invention as described in claim 6, further comprising the test strip defining a first layer and a second strip defining a second layer sandwiching opposites sides of the diaper body which further integrates a detection material constructed to react to a chemical conduit established between the layers.

10. An infection diagnosing diaper adapted to being worn by a patient, comprising:
a urine reactant incorporated into a test strip which is integrated in the diaper for indicating the existence of a positive condition not limited to diagnosing of a urinary tract infection;
the test strip defining a first layer and a second strip defining a second layer sandwiching opposites sides of the diaper body which further integrates a detection material constructed to react to a chemical conduit established between the layers; and
the urine reactant including at least one chemical agent or indicator and which, upon coming into contact with urine issued by the patient and triggering the positive condition, alerting a care provider of the condition.

11. The invention as described in claim 10, further comprising said test strip being integrated into either an interior or exterior of a body of the diaper.

12. The invention of claim 10 said test strip being positioned in extending fashion along a midsection of the diaper.

13. The invention as described in claim 10, said output further including a colorant which is released by instruction of the test strip in order to notify the wearer or care giver.
